(19) United States
(12) Patent Application Publication
(10) Pub. No.: US 2023/0288430 A1
(43) Pub. Date: Sep. 14, 2023

(54) METHOD FOR DETECTING INFLAMMATION-RELATED PLATELET ACTIVATION

(71) Applicants: Etablissement Français du Sang, LA PLAINE SAINT DENIS (FR); UNIVERSITE JEAN MONNET SAINT ETIENNE MAISON DE L'UNIVERSITE 10 RUE TREFILERIE 42100 SAINT-ETIENNE, SAINT-ETIENNE (FR)

(72) Inventors: Fabrice COGNASSE, SAINT-ETIENNE (FR); Thi Kim Anh NGUYEN-PEYRE, BUSSY SAINT GEORGES (FR); Olivier GARRAUD, PARIS (FR); Hind HAMZEH-COGNASSE, SAINT-ETIENNE (FR)

(21) Appl. No.: 17/923,161

(22) PCT Filed: May 4, 2021
(86) PCT No.: PCT/EP2021/061691

§ 371 Date: Nov. 3, 2022

(30) Foreign Application Priority Data
May 5, 2020 (FR) 20 04447

(51) Int. Cl. G01N 33/68

(52) U.S. Cl. CPC G01N 33/6893; G01N 2333/70596; G01N 2333/912; G01N 2333/523; G01N 2333/70578; G01N 2800/52; G01N 2800/56; G01N 2800/7095

(57) ABSTRACT

The present invention relates to a method for detecting inflammation-related platelet activation, comprising measuring the quantity, concentration and/or the proportion of seven specific biomarkers representative of 47 intra-platelet, soluble and membrane molecules, in a biological sample. Said panel of seven biomarkers is particularly useful for implementing a method of diagnosing inflammation-related platelet activation in an individual, a method for monitoring the efficacy of a curative or preventive treatment of an inflammatory disease in an individual and/or a method for monitoring the development of an inflammatory disease associated with inflammation-related platelet activation in an individual.

The present invention also relates to a kit for detecting inflammation-related platelet activation, as well as the use thereof.

### TECHNICAL FIELD

The present invention relates to the detection of an inflammation-related platelet activation.

### PRIOR ART

Blood platelets are anucleate cells which play a major role in the processes of primary hemostasis. Platelets also participate in the inflammatory process, covering the detection of the danger signal, the release of soluble factors and the interaction with leukocyte and endothelial cells.

Platelets contain in the □ and α granules thereof immunomodulatory factors such as growth factors, cytokines (CK), chemokines (CH) and Damage Associated Molecular Patterns (DAMPs) which are released during the activation thereof. Such first observations allow the link to be established between the thrombotic and inflammatory events observed in many cardiovascular pathologies (endocarditis, atherosclerosis). Among all the released factors, the CD40-Ligand (CD40L) is a fundamental molecule in the activation of leukocytes and endothelial cells.

The involvement of platelets in the immune response was also confirmed by the fact that same express functional Toll Like Receptors (TLRs), key receptors in the recognition of patterns conserved on the surface of pathogens. Because of such feature, platelets could then be considered sentinels of innate immunity. Platelets also interact with leukocytes through either direct or indirect contact via fibrinogen.

All observations concerning the involvement of platelets in innate immunity have mainly been made individually. However, the observations revealed that platelets have the arsenal required for participating in complex mechanisms such as inflammation. From a molecular point of view, inflammation involves immunomodulatory, soluble (chemokines, cytokines) or cellular (selectins, integrins) molecules, leading to the extravasation of leukocytes, so that leukocytes can exercise the inflammatory function thereof directly on the mucose.

Thereby, many studies associate inflammatory pathologies with an either direct or indirect dysfunction of platelet physiology.

It would, therefore, be useful to have means for easily detecting inflammation-related platelet activation.

### DETAILED DESCRIPTION

Surprisingly, the Inventors have demonstrated a panel of seven biomarkers which are necessary and sufficient for effectively detecting inflammation-related platelet activation.

The panel of biomarkers was identified among 47 soluble or membrane intra-platelet molecules, selected beforehand by the inventors from a much wider set of molecules potentially associated with platelet activation.

For this purpose, in vitro experimental conditions approaching the conditions found in the bloodstream, were used. Platelet-rich plasmas (PRP) from 10 donors were either or not subjected to six different inflammation-related stimulation conditions: a PAR-1 agonist, a PAR-4 agonist, ADP (adenosine diphosphate), collagen, sCD40L (soluble CD40 ligand) or fibrinogen.

A biomathematical analysis of the data obtained for the 47 biological parameters either or not under the 6 conditions of stimulation, has thus led to the identification of the following seven molecules, qualified as biomarkers, which alone can detect inflammation-related platelet activation: AKT, PKC, CD62P, CD63, RANTES, TSLP and CD40 ligand.

The biomathematical analysis also identified specific biomarkers of the activation of a given intra-platelet signaling pathway.

The demonstration of the panel of seven biomarkers thus allows inflammation-related platelet activation to be specifically detected the first time.

The panel of seven biomarkers advantageously limits the number of molecules tested for characterizing platelet activation.

The measurement of the quantity, concentration and/or the proportion of said seven biomarkers in a physiopathological context thus advantageously allows inflammation-related platelet activation to be detected.

A first subject matter of the invention is a method for detecting inflammation-related platelet activation, comprising measuring the quantity, concentration and/or the proportion of AKT, PKC, CD62P, CD63, RANTES, TSLP and CD40 ligand in a biological sample.

Another subject matter of the invention is a method of diagnosis of inflammation-related platelet activation in an individual, comprising:
a) measuring the quantity, concentration and/or the proportion of AKT, PKC, CD62P, CD63, RANTES, TSLP and CD40 ligand biomarkers in a biological sample of said individual,
b) comparing the results obtained during the step a) with a corresponding standard control value, and
c) deducing from the above if the individual has inflammation-related platelet activation.

Step c) of the method of diagnosis for inflammation-related platelet activation as defined above can include deducing therefrom whether a specific intra-platelet signaling pathway is activated.

Another subject matter of the invention is a method for monitoring the efficacy of a curative or preventive treatment of an inflammatory disease in an individual, comprising:
a) measuring the quantity, concentration and/or the proportion of AKT, PKC, CD62P, CD63, RANTES, TSLP and CD40 ligand biomarkers in a biological sample of said individual at a time t during said treatment,
b) comparing the results obtained during the step a) with a corresponding standard control value and/or with a corresponding value obtained before the start of said treatment or at a time during the treatment, which is earlier than time t,
c) deducing therefrom whether the treatment is effective, and
d) optionally, repeating the steps a) to c).

Another subject matter of the invention is a method for monitoring the evolution of an inflammatory disease associated with inflammation-related platelet activation in an individual, comprising:
a) measuring the quantity, concentration and/or the proportion of AKT, PKC, CD62P, CD63, RANTES, TSLP and CD40 ligand biomarkers in a biological sample of said individual at times t1 and t2, separated in time,
b) comparing the results at time t1 and time t2 obtained during the step a),
c) deducing therefrom whether the inflammatory disease evolves favorably, and
d) optionally, repeating the steps a) to c).

Another subject matter of the invention is a stratification method for an individual suffering from or at risk of suffering from an inflammatory disease, within a class of individuals with inflammation-related platelet activation or within a class of individuals without any inflammation-related platelet activation, comprising:
a) measuring the quantity, concentration and/or the proportion of AKT, PKC, CD62P, CD63, RANTES, TSLP and CD40 ligand biomarkers in a biological sample of said individual,
b) comparing the results obtained during the step a) with a corresponding standard control value, and
c) deducing therefrom whether the individual belongs to the class of individuals with inflammation-related platelet activation or to the class of individuals without inflammation-related platelet activation.

Within the methods as defined above, the inflammatory disease is e.g. selected from the group consisting of atherosclerosis, pulmonary inflammation, rheumatoid arthritis (RA), inflammatory bowel disease (IBD), sepsis, severe sepsis, septic shock, cancer and combinations thereof.

Within the methods as defined above, the biological sample is preferentially a platelet-rich plasma sample.

Another subject matter of the invention is a kit suitable for detecting inflammation-related platelet activation comprising means for detecting at least seven biomarkers, characterized in that said at least seven biomarkers are AKT, PKC, CD62P, CD63, RANTES, TSLP and CD40 ligand.

Another subject matter of the invention relates to the use of the kit as defined above for the detection of inflammation-related platelet activation.

Another subject matter of the invention relates to the use of a panel of biomarkers comprising AKT, PKC, CD62P, CD63, RANTES, TSLP and CD40 ligand for the detection of inflammation-related platelet activation.

Biological Sample

The detection of inflammation-related platelet activation is evaluated in a biological sample.

The biological sample is a sample comprising platelets.

“Platelets” refers herein to blood platelets and culture platelets.

“Blood platelets”, also called “thrombocytes” refer herein to cells without a nucleus, circulating in the blood. Blood platelets are formed in the bone marrow from the fractionation of megakaryocytes.

“Culture platelets” refer herein to platelets obtained from megakaryocytes cultured in vitro. Megakaryocytes then preferentially come from a line of immortalized megakaryocytes.

A biological sample can include blood platelets or culture platelets.

A biological sample comprising blood platelets can be a sample from one or more individuals.

A biological sample comprising culture platelets can be a sample of culture medium.

A biological sample is preferentially a sample from one or more individuals.

The individual(s) are e.g. as defined hereinafter in the “individual” section.

A preferred biological sample according to the invention is a platelet-rich plasma (PRP) sample.

The platelet-rich plasma sample can be obtained by any suitable method well known to a person skilled in the art.

A platelet-rich plasma sample can be obtained e.g. by centrifugation of a blood sample, e.g. at 192 g for 5 to 10 minutes, and by recovery of the upper phase corresponding to the platelet-rich plasma.

A platelet-rich plasma sample can come from an individual, a mixture of platelet-rich plasmas from at least two individuals, or a platelet-rich plasma obtained from a mixture of blood from at least two individuals.

A platelet-rich plasma sample comes preferentially from an individual.

Individual

An individual is preferentially a mammal, e.g. either a human individual or a non-human mammal, such as a cat, a dog, a monkey, a rabbit, a mouse or a rat.

A human individual can be either a man or a woman of any age, such as an infant, a child, a teenager, an adult or an elderly person.

An individual is preferentially an individual suffering from or at risk of suffering from an inflammatory disease.

An individual at risk of suffering from an inflammatory disease is e.g. an individual with a family history of inflammatory disease, having already suffered from at least one inflammatory disease, having genetic, metabolic and/or lifestyle-related risk factors and/or presenting at least one symptom which can be a precursor to an inflammatory disease.

An inflammatory disease is in particular as defined hereinafter in the section “inflammatory disease”.

Inflammatory Disease

An inflammatory disease, also called inflammatory pathology, can be any disease resulting from an abnormal reaction of the immune system.

An inflammatory disease can be an autoinflammatory disease, an autoimmune disease or an inflammatory disease of undetermined origin.

Inflammatory pathologies are defined by a tissue aggression due to a deregulation in the immune system. The latter further include atherosclerosis, rheumatoid arthritis, pulmonary inflammation, inflammatory bowel disease, sepsis, severe sepsis, septic shock or cancer.

The inflammatory disease is thus preferentially selected from the group consisting of atherosclerosis, rheumatoid arthritis, pulmonary inflammation, inflammatory bowel disease, sepsis, severe sepsis, septic shock, cancer and combinations thereof.

Atherosclerosis (also called arteriosclerosis) is a disease affecting the arteries and characterized by the appearance of atheromas on the inner wall of the arteries.

Rheumatoid arthritis (or RA) is a systemic disease of the connective tissue characterized by chronic joint inflammation which develops by episodes.

Pulmonary inflammation includes inflammatory lung disease and/or pulmonary allergy.

Inflammatory lung disease includes Acute Lung Injury or ALI, also known as “moderate ARDS”, Acute Respiratory Distress Syndrome (ARDS), Transfusion Related Acute Lung Injury (TRALI).

Pulmonary allergy, also called respiratory allergy, is a disease which alternates between an acute phase (also called an asthma attack) and a chronic phase.

Inflammatory bowel disease (IBD) can be selected from Crohn's disease and ulcerative colitis.

An excessive inflammatory reaction, in response to infection, causes sepsis and the most severe forms thereof, severe sepsis and septic shock.

The cancer cell will cause inflammation and invade neighboring cells so as to create a climate conducive to the growth of nearby cells. This is how, little by little, a cancerous tumor is born. Concretely, the cancer cell uses inflammation to progress.

Biomarkers

In particular, the present invention provides a panel of seven biomarkers useful for detecting inflammation-related platelet activation. The seven biomarkers are CD62P, CD63, AKT, PKC, RANTES, TSLP and CD40 ligand.

CD62P and CD63 are membrane proteins.

CD62P (also known as P selectin, GMP-140 or PADGEM) is a glycoprotein encoded by the SELP gene in humans. Same is expressed on the surface of blood platelets and activated endothelial cells. CD62P functions as a cell adhesion molecule on the surface of activated endothelial cells and activated platelets.

CD63 is a protein encoded by the CD63 gene in humans.

AKT and PKC are intracellular proteins.

AKT (also known as AKT1 or protein kinase B) is a serine-threonine kinase protein encoded in humans by the Akt1 gene.

PKC (also known as protein kinase C) is a serine/threonine kinase protein.

RANTES, TSLP and CD40 ligand are soluble proteins.

RANTES (also called CCL5) is a protein encoded in humans by the CCL5 gene.

TSLP (also known as thymic stromal lymphopoietin) is a protein encoded in humans by the TSLP gene.

CD40 ligand (also called CD40L or CD154) is a transmembrane glycoprotein member of the TNF (Tumor Necrosis Factor) superfamily, which has a membrane-type form after activation, then a soluble form after cleavage.

Method for Detecting Inflammation-Related Platelet Activation

Having a method for detecting inflammation-related platelet activation can be particularly useful in many applications, such as research and development and/or in the medical field.

In research and development e.g., the detection of inflammation-related platelet activation can be used to test inflammation-related platelet activation, e.g. to test the ability of a molecule to induce or inhibit inflammation-related platelet activation and/or to develop an inflammation-related platelet activation model or to test inflammation-related platelet activation.

In the medical field e.g., the detection of inflammation-related platelet activation can make it possible to establish a diagnosis or to help establishing a diagnosis for an individual suffering from or at risk of suffering from an inflammatory disease, to subclassify an individual suffering from or likely to suffer from inflammatory disease in the category “individual with inflammation-related platelet activation” or in the category “individual without inflammation-related platelet activation”, to monitor the evolution of inflammatory disease over time, and/or monitor the efficacy of a curative or preventive treatment for an inflammatory disease.

The subject matter of the present invention is thus more particularly a method, in particular, an in vitro method, for detecting inflammation-related platelet activation, comprising measuring the quantity, concentration and/or the proportion of the AKT, PKC, CD62P, CD63, RANTES, TSLP and CD40 ligand biomarkers in a biological sample.

“Proportion of a biomarker” refers e.g. to the percentage of cells expressing said biomarker in a cell population, in particular in a population of platelets. The measurement of the proportion of a biomarker is then a measurement of the expression of said biomarker.

The biological sample is in particular as defined hereinabove.

The method as defined above can comprise, in particular:
a) measuring the quantity, concentration and/or the proportion of AKT, PKC, CD62P, CD63, RANTES, TSLP and CD40 ligand biomarkers in a biological sample,
b) optionally, comparing the results obtained during the step a) with a corresponding standard control value, and
c) optionally, deducing therefrom whether platelet activation is present in the biological sample.

Step a)

During the step a), the quantity, concentration and/or the proportion of AKT, PKC, CD62P, CD63, RANTES, TSLP and CD40 ligand biomarkers is measured in a biological sample.

The measurement of the quantity, concentration and/or the proportion of a biomarker in a biological sample can be performed directly in the biological sample or after treating the biological sample.

“Biological sample treatment” refers herein to at least one treatment step, e.g. selected from the group consisting of a centrifugation, a freezing and then a thawing cycle, a lysis, a suspension in a buffer and a washing.

Depending on the biomarker, the measurement of the quantity, concentration and/or the proportion of said biomarker can thus be measured directly in the biological sample or after the treatment of the biological sample, e.g. in a platelet supernatant, in a platelet lysate, in a platelet lysate supernatant and/or a platelet pellet, said platelet pellet preferentially being resuspended in a buffer.

“Platelet supernatant” refers herein to the supernatant obtained after the centrifugation of the biological sample, e.g. at 490 g for 10 minutes.

“Platelet pellet” refers herein to the pellet obtained after the centrifugation of the biological sample, e.g. at 490 g for 10 minutes.

The platelet pellet can be resuspended in a buffer, such as calcium-free magnesium-free “PBS” (Phosphate-Buffered Saline) or a “Tyrode” buffer.

“Platelet lysate” refers herein to the lysate obtained after the lysis of the platelets.

The platelet lysate can e.g. be obtained by the centrifuging of the biological sample so as to obtain a platelet pellet, then suspending at least a portion of said platelet pellet in a lysis buffer. Alternatively, the platelet lysate can be obtained by freezing, then thawing the platelets, e.g. by freezing, then thawing the biological sample, at least a part of the platelet pellet and/or at least a part of said platelet pellet resuspended in a buffer, such as calcium-free magnesium-free PBS (Phosphate-Buffered Saline) or a Tyrode buffer.

“Platelet lysate supernatant” refers herein to the supernatant obtained after the centrifugation of the platelet lysate, e.g. at 490 g for 10 min.

(i) CD62P and CD63

The measurement of the quantity, concentration and/or the proportion of the CD62P and CD63 biomarkers can be performed by any method well known to a person skilled in the art, for measuring the quantity, concentration and/or the proportion of a membrane protein.

The quantity, concentration and/or the proportion of the CD62P and CD63 biomarkers is e.g. measured by flow cytometry or related techniques for counting and measuring cell properties (such as size, cell content, fluorescence, etc.).

The step a) as defined hereinabove comprises e.g. measuring of the proportion of the CD62P biomarker and/or the CD63 biomarker in percentage and/or measuring the quantity of the CD62P biomarker and/or the CD63 biomarker in mean fluorescence intensity (MFI).

The mean fluorescence intensity is preferentially measured by flow cytometry within the window corresponding to platelets. The window corresponding to the platelets is e.g. defined by using the marker CD41 which is characteristic of the platelets.

The proportion of the biomarker, in particular CD62P or CD63, corresponds to the percentage of cells expressing said biomarker among the cells within the window corresponding to platelets. The proportion of the biomarker is preferentially obtained by flow cytometry.

The step a) as defined hereinabove preferentially comprises measuring the proportion of the biomarkers CD62P and CD63 as a percentage.

The quantity, concentration and/or the proportion of the biomarker CD62P and/or CD63 can be measured directly in the biological sample or in part of the platelet pellet, preferentially resuspended in a buffer, such as calcium-free magnesium-free Phosphate Buffered Saline (PBS) or a Tyrode buffer.

(ii) AKT and PKC

The measurement of the quantity, concentration and/or the proportion of AKT and PKC biomarkers can be performed by any method well known to a person skilled in the art, for measuring the quantity, concentration and/or the proportion of an intracellular protein.

The quantity, concentration and/or the proportion thereof is e.g. measured in the platelet lysate, in the platelet lysate supernatant and/or in the platelet supernatant.

(iii) RANTES, TSLP and CD40 ligand

The measurement of the quantity, concentration and/or the proportion of the RANTES, TSLP and CD40 ligand biomarkers can be performed by any method well known to a person skilled in the art, for measuring the quantity, concentration and/or the proportion of a soluble protein.

The quantity, concentration and/or the proportion is e.g. measured in the supernatant of the platelets.

Any type of test for specifically detecting and/or quantifying the biomarker present in a liquid medium (e.g. biological sample, platelet pellet resuspended in buffer, platelet lysate, platelet lysate supernatant or platelet supernatant) can be used, such as an immunological test (e.g. a multiplexed immunological test, preferentially run on spots or on beads), or Western blot.

Preferentially, the step a) as defined hereinabove comprises the measurement of the concentration of AKT, PKC, RANTES, TSLP and CD40 ligand, e.g. by weight through the number of platelets (in particular after having performed a platelet count, e.g. of the NFS type ([Numeration et Formule Sanguine]=complete blood count). The concentration is expressed e.g. in ng/109 platelets.

Thus, the step a) as defined hereinabove can comprise:
centrifuging at least a part of the biological sample so as to obtain a platelet pellet and a platelet supernatant,
optionally lysing a part of the platelet pellet so as to obtain a platelet lysate, e.g. by means of a lysis buffer or by freezing and thawing, and optionally centrifuging said platelet lysate so as to obtain a platelet lysate supernatant,
measuring the quantity, concentration and/or the proportion of:
   AKT and PKC biomarkers in said platelet supernatant, said platelet lysate and/or in said platelet lysate supernatant,
   RANTES, TSLP and CD40 ligand biomarkers in said platelet supernatant, and
   CD62P and CD63 biomarkers in a part of the biological sample and/or in a part of said platelet pellet, optionally resuspended in a buffer.

Step b)

During the step b), the results obtained during the step a) are compared with a corresponding standard control value.

The results obtained during the step a) are the quantity, concentration and/or the proportion of the biomarkers AKT, PKC, CD62P, CD63, RANTES, TSLP and CD40 ligand in the biological sample.

The term “corresponding” in the expression “corresponding standard control value” means that, for every biomarker, the quantity, concentration and/or the proportion thereof as measured in the biological sample, is compared with a standard control value for that biomarker.

If the quantity of the biomarker is what is measured in the biological sample, the standard control value is a quantity.

If the concentration of the biomarker is what is measured in the biological sample, the standard control value is a concentration.

If the proportion of the biomarker is what is measured in the biological sample, the standard control value is a proportion.

A standard control value, also referred to as the reference value of a biomarker X can be e.g. the mean, the maximum threshold value and/or the minimum threshold value of the quantity, the concentration or the proportion of the biomarker X as measured in the samples taken from a reference population, preferentially consisting of healthy individuals or individuals suffering from an inflammatory disease before treatment.

The values measured in the reference population are obtained using a measurement method similar to the method used for the biological sample.

Two measurement methods are similar if, when used on the same sample, same give an identical or similar result. In this respect, the calculation of the coefficient of variation (CV) is an important measure of the reliability of the method which is acceptable if CV<20%.

The minimum threshold value and the maximum threshold value define a reference interval. The reference interval is generally defined so as to include 95% of the values obtained in the reference population.

“Reference population” refers to a population of individuals wherein the biomarker X is measured.

The reference population can comprise at least 5 individuals, e.g. 10 individuals or at least 10 individuals, e.g. at least 50 individuals or at least 100 individuals.

The reference population preferentially consists of healthy individuals, i.e. individuals with a good general state of health.

The standard control value of a biomarker X can be the mean, the maximum threshold value and/or the minimum threshold value of the quantity, the concentration or the proportion of the biomarker X as measured in samples taken from a reference population of healthy individuals, samples which either were or were not stimulated with a platelet stimulator.

The platelet stimulator is e.g. selected from the group consisting of a PAR-1 agonist (e.g., TRAP SEQ ID NO: 1), a PAR-4 agonist (e.g. the peptide SEQ ID NO: 2), ADP, collagen, sCD40L (soluble ligand of CD40) or fibrinogen.

A standard control value can e.g. correspond to the mean of the values obtained in the samples from 10 healthy individuals, without stimulation of the platelets.

A standard control value can thus further correspond to the mean of the values obtained in the samples coming from 10 healthy individuals, with stimulation of the platelets, in particular by using a platelet stimulator selected from the group consisting of a PAR-1 agonist (e.g. TRAP SEQ ID NO: 1), a PAR-4 agonist (e.g. the peptide SEQ ID NO: 2), ADP, collagen, sCD40L (soluble ligand of CD40) or fibrinogen.

Alternatively, the reference population can consist of individuals with inflammation-related platelet activation, preferentially with the same inflammatory disease.

Alternatively, the standard control value can be a set value, e.g. set by an administration, said value not necessarily corresponding to the mean, minimum threshold value or maximum threshold value of the values of a given biomarker in a reference population.

A standard control value corresponding to a population of healthy individuals (in particular a set value or a value obtained from samples not subject to platelet activation and coming from a reference population consisting of healthy individuals) is qualified as an NA (not activated) standard control value.

A standard control value corresponding to a population of individuals with inflammation-related platelet activation (in particular a set value, a value obtained from samples subject to platelet activation and from a reference population consisting of healthy individuals or a value obtained from samples coming from a reference population consisting of individuals with inflammation-related platelet activation) is qualified as an A (activated) standard control value.

A standard control value A is necessarily different from an NA standard value.

For AKT, PKC, CD62P, CD63, RANTES, TSLP and CD40 ligand markers, the A standard control value is necessarily greater than the NA standard value.

If the quantity, concentration and/or the proportion of the biomarker AKT, PKC, CD62P, CD63, RANTES, TSLP or CD40 ligand is:
greater than or equal to an A standard control value (in particular a minimum threshold value), and/or
greater than an NA standard control value (in particular a maximum threshold value), the biomarker is called activated.

If the quantity, concentration and/or the proportion of the biomarker AKT, PKC, CD62P, CD63, RANTES, TSLP or CD40 ligand is:
less than an A standard control value (in particular a minimum threshold value), and/or
less than or equal to an NA standard control value (in particular a maximum threshold value), the biomarker is described as non-activated.

Step c)

During the step c), either the presence or the absence of inflammation-related platelet activation in the biological sample, is deduced.

In particular, if the quantity, concentration and/or the proportion of at least three of said biomarkers is greater than a corresponding NA standard control value (in particular the maximum threshold value or a set value), the presence of inflammation-related platelet activation in the biological sample can be deduced therefrom.

In particular, if the quantity, concentration and/or the proportion of at least five of said biomarkers is less than or equal to the corresponding NA standard control value (in particular the maximum threshold value or a set value), the absence of inflammation-related platelet activation in the biological sample is deduced therefrom.

In particular, if the quantity, concentration and/or the proportion of at least three of said biomarkers is greater than or equal to a corresponding A standard control value (in particular the minimum threshold value or a set value), the presence of inflammation-related platelet activation in the biological sample is deduced therefrom.

In particular, if the quantity, concentration and/or the proportion of at least five of the said biomarkers is less than the corresponding A standard control value (in particular the minimum threshold value or a set value), the absence of inflammation-related platelet activation in the biological sample is deduced therefrom.

Method of Diagnosis for Inflammation-Related Platelet Activation

A further subject matter of the present invention is a method of diagnosis for inflammation-related platelet activation in an individual, comprising:
a) measuring the quantity, concentration and/or the proportion of AKT, PKC, CD62P, CD63, RANTES, TSLP and CD40 ligand biomarkers in a biological sample of said individual,
b) comparing the results obtained during the step a) with a corresponding standard control value, and
c) deducing from the above if the individual has inflammation-related platelet activation.

A biological sample is a sample from an individual, in particular as defined above in the “Sample” section.

In particular, the individual is as defined above in the “individual” section.

An individual is preferentially an individual suffering from or at risk of suffering from an inflammatory disease, in particular an inflammatory disease as defined above in the section “inflammatory disease”.

The steps a) and b) are in particular as defined above in the section “Method for detecting platelet inflammation”.

During the step c), it is deduced therefrom whether the individual has an inflammation-related platelet activation.

In particular, if the quantity, concentration and/or the proportion of at least three of said biomarkers is greater than a corresponding NA standard control value (in particular the maximum threshold value or a set value), it is deduced therefrom that the individual has an inflammation-related platelet activation.

In particular, if the quantity, concentration and/or the proportion of at least five of the said biomarkers is less than or equal to the corresponding NA standard control value (in particular the maximum threshold value or a set value), it is deduced therefrom that the individual does not have an inflammation-related platelet activation.

In particular, if the quantity, concentration and/or the proportion of at least three of said biomarkers is greater than or equal to a corresponding A standard control value (in particular the minimum threshold value or a set value), it is deduced therefrom that the individual has an inflammation-related platelet activation.

In particular, if the quantity, concentration and/or the proportion of at least five of said biomarkers is less than the corresponding A standard control Value (in particular the minimum threshold value or a set value), it is deduced therefrom that the individual does not have an inflammation-related platelet activation.

The step c) can comprise deducing therefrom whether a specific intra-platelet signaling pathway is activated.

E.g.
if the seven biomarkers AKT, PKC, CD62P, CD63, RANTES, TSLP and CD40 ligand are activated, the PAR-4 signaling pathway is activated,
If the biomarkers AKT, CD62P, CD63, RANTES and CD40 are activated, but the biomarkers PKC and TSLP are not activated, the PAR-1 signaling pathway is activated.
If the biomarkers AKT, CD40L, and TSLP biomarkers are activated, but the biomarkers PKC, CD62P, CD63, and RANTES are not activated, the fibrinogen signaling pathway is activated.
if the biomarker CD40L is not activated, but at least three biomarkers are activated, the sCD40L signaling pathway is activated,
If the biomarkers AKT, PKC, CD62P, RANTES and CD40 ligand are activated, but the biomarkers TSLP and CD63 are not activated, the ADP signaling pathway is activated, or
if the biomarkers AKT, CD62P, RANTES, TSLP and CD40 ligand are activated, but not PKC, the collagen signaling pathway is activated.

A Method for Preventing and/or Treating Inflammatory Disease Associated with Inflammation-Related Platelet Activation

A further subject matter of the present invention is a method for preventing and/or treating an inflammatory disease associated with inflammation-related platelet activation in an individual, comprising:
a) measuring the quantity, concentration and/or the proportion of AKT, PKC, CD62P, CD63, RANTES, TSLP and CD40 ligand biomarkers in a biological sample of said individual,
b) comparing the results obtained during the step a) with a corresponding standard control value,
c) deducing therefrom whether the individual has an inflammation-related platelet activation,
d) if the individual has an inflammation-related platelet activation, administering antiplatelet therapy to said individual.

In particular, the individual is as defined above in the “individual” section.

An inflammatory disease is e.g. as defined hereinabove in the section “inflammatory disease”.

A biological sample is a sample from an individual, in particular as defined above in the “Sample” section.

A biological sample is preferentially a platelet-rich plasma sample.

The steps a) to c) are as defined above in the section “Diagnosis Procedure for inflammation-related platelet activation”.

Antiplatelet therapy can be a treatment which decreases or inhibits platelet activity and/or a treatment which decreases or inhibits at least one of the activated biomarkers.

Method for Monitoring the Efficacy of a Curative or Preventive Treatment for an Inflammatory Disease

Another subject matter of the present invention is a method for monitoring the efficacy of a curative or preventive treatment of an inflammatory disease in an individual, comprising:
a) measuring the quantity, concentration and/or the proportion of AKT, PKC, CD62P, CD63, RANTES, TSLP and CD40 ligand biomarkers in a biological sample of said individual at a time t during said treatment,
b) comparing the results obtained during the step a) with a corresponding standard control value and/or with a corresponding value obtained before the start of said treatment or at a time during the treatment, which is earlier than time t, and
c) deducing therefrom whether the treatment is effective,
d) optionally, repeating the steps a) to c).

An inflammatory disease is e.g. as defined hereinabove in the section “inflammatory disease”.

A biological sample is a sample from an individual, in particular as defined above in the “Sample” section.

A biological sample is preferentially a platelet-rich plasma sample.

A curative or preventive treatment can comprise curative or preventive treatment of inflammatory disease and/or curative or preventive treatment of an inflammation-related platelet activation.

The curative or preventive treatment of inflammation-related platelet activation is e.g. antiplatelet therapy as defined hereinabove in the section “Method for preventing and/or treating an inflammatory disease associated with inflammation-related platelet activation”.

The curative or preventive treatment of inflammatory disease can e.g. comprise the administration of corticosteroids, immunomodulators, 5-aminosalicylates, bronchodilators and/or non-steroidal anti-inflammatory drugs.

The characteristics of the steps a) and b) are generally as defined for the steps a) and b) as defined in the section “Method for detecting platelet inflammation”.

Within the method for monitoring the efficacy of a treatment defined above, step a) includes measuring the quantity, concentration and/or the proportion of the biomarkers AKT, PKC, CD62P, CD63, RANTES, TSLP and CD40 ligand in a biological sample of said individual at a time t during treatment.

In other words, the measurement is performed in a biological sample coming from a sample taken at time t.

During the step b), the results obtained during the step a) are compared with a corresponding standard control value and/or with a corresponding value obtained before the start of said treatment or at a time during the treatment which precedes time t.

“Corresponding value obtained before the start of said treatment” refers to the quantity, concentration and/or the proportion of the corresponding biomarker as measured in a biological sample of said patient, said sample having been taken before the start of the treatment.

“Corresponding value obtained at a time during the treatment which precedes time t” refers to the quantity, concentration and/or the proportion of the corresponding biomarker as measured in a biological sample of said patient, said sample having been taken after the start of the treatment, but before time t.

During the step c), it is deduced therefrom whether the treatment is effective.

The treatment is effective e.g. if the inflammation-related platelet activation decreases or disappears.

The treatment is effective e.g. if, for at least one biomarker activated prior to the initiation of the treatment or at a point in time during the treatment prior to time t, the quantity, concentration and/or the proportion of the biomarker as measured at time t is less than a corresponding value obtained before the start of said treatment or at a time during the treatment which precedes time t.

The treatment is effective e.g. if at least one biomarker is no longer activated at time t, whereas it was activated before the start of said treatment and/or at a time during the treatment which is prior to time t.

The treatment is not effective e.g. if, for at least one biomarker activated before the start of the treatment or at a point in time during the treatment which precedes time t, the quantity, concentration and/or the proportion of the biomarker as measured at time t is greater than a corresponding value obtained before the start of said treatment or at a time during the treatment which precedes time t.

The treatment is not effective e.g. if at least one biomarker is activated at time t, whereas it was not activated before the start of said treatment and/or at a time during treatment which is prior to time t.

If the treatment is not effective, the method can comprise the administration of a new or supplementary treatment.

The method can comprise a step d) wherein steps a) to c) are repeated, in particular whether the treatment is effective or not.

The steps a) to c) can be repeated once, twice, three times or at least three times.

The steps a) to c) can be repeated, e.g., until the individual is cured or the inflammatory disease is stabilized.

The steps a) and c) can be repeated at regular or non-regular time intervals.

The period between repetitions can be e.g. at least one week, at least two weeks, at least three weeks, at least four weeks, at least two months, at least three months, at least four months, at least five months, six months or at least six months, e.g. a year.

The method for monitoring the efficacy of a curative or preventive treatment can be used each time a new or supplementary treatment is initiated.

Method for Monitoring the Development of an Inflammatory Disease Associated with an Inflammation-Related Platelet Activation

A further subject matter of the present invention is a method for monitoring the evolution of an inflammatory disease associated with an inflammation-related platelet activation in an individual, comprising:
a) measuring the quantity, concentration and/or the proportion of AKT, PKC, CD62P, CD63, RANTES, TSLP and CD40 ligand biomarkers in a biological sample of said individual at times t1 and t2, separated in time,
b) comparing the results obtained at time t1 and time t2 during the step a), and
c) deducing therefrom whether the inflammatory disease evolves favorably,
d) optionally, repeating the steps a) to c).

An inflammatory disease is e.g. as defined hereinabove in the section “inflammatory disease”.

A biological sample is a sample from an individual, in particular as defined above in the “Sample” section.

A biological sample is preferentially a platelet-rich plasma sample.

The characteristics of the steps a) and b) are generally as defined for the steps a) and b) as defined in the section “Method for detecting platelet inflammation”.

Within the method for monitoring the evolution of an inflammatory disease as defined above, the step a) comprises measuring the quantity, concentration and/or the proportion of the biomarkers AKT, PKC, CD62P, CD63, RANTES, TSLP and CD40 ligand in a biological sample of said individual at two times t1 and t2 separated in time.

In other words, the measurement is performed in a biological sample coming from a sample taken at time t1, as well as in a biological sample coming from a sample taken at time t2, time t1 being prior to time t2.

The time interval between t1 and t2 is e.g. at least one week, at least two weeks, at least three weeks, at least four weeks, at least two months, at least three months, at least four months, at least five months, six months or at least six months, e.g. a year.

During the step b), the results obtained during the step a), at time t1 and at time t2, are compared with each other.

During the step c), it is deduced therefrom whether the inflammatory disease evolves favorably.

An inflammatory disease evolves favorably e.g. if the inflammation-related platelet activation decreases or disappears between t1 and t2.

The inflammatory disease evolves favorably e.g. if, for at least one biomarker activated at time t1, the quantity, concentration and/or the proportion of the biomarker decreases between time t1 and t2 and/or if at least one biomarker activated at time t1 is no longer activated at time t2.

The inflammatory disease does not evolve favorably e.g. if, for at least one biomarker activated at time t1, the quantity, concentration and/or the proportion of the biomarker increases between time t1 and t2 and/or if at least one biomarker is activated at time t2, whereas same was not activated at time t1.

The method can comprise a step d) wherein the steps a) to c) are repeated, in particular whether or not the inflammatory disease evolves favorably.

The steps a) to c) can be repeated once, twice, three times or at least three times.

The steps a) to c) can be repeated, e.g., until the individual is cured or the inflammatory disease is stabilized.

The steps a) and c) can be repeated at regular or non-regular time intervals.

The period between repetitions can be e.g. at least one week, at least two weeks, at least three weeks, at least four weeks, at least two months, at least three months, at least four months, at least five months, six months or at least six months, e.g. a year.

Stratification Method for an Individual Suffering from or at Risk of Suffering from an Inflammatory Disease

A further subject matter of the invention is a stratification method for an individual suffering from or at risk of suffering from an inflammatory disease, within a class of individuals with inflammation-related platelet activation or within a class of individuals without any inflammation-related platelet activation, comprising:
a) measuring the quantity, concentration and/or the proportion of AKT, PKC, CD62P, CD63, RANTES, TSLP and CD40 ligand biomarkers in a biological sample of said individual,
b) comparing the results obtained during the step a) with a corresponding standard control value, and
c) deducing therefrom whether the individual is in the category of individuals with an inflammation-related platelet activation or in the category of individuals without any inflammation-related platelet activation.

A biological sample is a sample from an individual, in particular as defined above in the “Sample” section.

An inflammatory disease is in particular as defined above in the section “inflammatory disease”.

The steps a) and b) are in particular as defined above in the section “Method for detecting platelet inflammation”.

In particular, if the quantity, concentration and/or the proportion of at least three of said biomarkers is greater than a corresponding NA standard control value (in particular the maximum threshold value or a set value), it is deduced therefrom that the individual is in the category of individuals with an inflammation-related platelet activation.

In particular, if the quantity, concentration and/or the proportion of at least five of the said biomarkers is less than or equal to the corresponding NA standard control value (in particular the maximum threshold value or a set value), it is deduced therefrom that the individual is in the category of individuals without any inflammation-related platelet activation.

In particular, if the quantity, concentration and/or the proportion of at least three of said biomarkers is greater than or equal to a corresponding A standard control value (in particular the minimum threshold value or a set value), it is deduced therefrom that the individual is in the category of individuals with an inflammation-related platelet activation.

In particular, if the quantity, concentration and/or the proportion of at least five of the said biomarkers is less than the corresponding A standard control value (in particular the minimum threshold value or a set value), it is deduced therefrom that the individual is in the category of individuals without any inflammation-related platelet activation.

Kit Suitable for the Detection of an Inflammation-Related Platelet Activation and the Use Thereof

A further subject matter of the present invention is a kit suitable for detecting an inflammation-related platelet activation comprising means for detecting at least seven biomarkers, characterized in that said at least seven biomarkers are AKT, PKC, CD62P, CD63, RANTES, TSLP and CD40 ligand.

The means of detection are, e.g., detection ligands specific for said biomarkers, optionally labeled.

The detection ligands specific for said biomarkers are preferentially antibodies.

The kit can comprise means for lysing platelets, e.g. a lysis buffer.

The present invention further relates to the use of a kit as defined above for the detection of inflammation-related platelet activation, in particular by the use of a method for detecting inflammation-related platelet activation as defined hereinafter.

Use of a Panel of Biomarkers

The present invention further relates to the use, in particular in vitro, of a panel of biomarkers comprising AKT, PKC, CD62P, CD63, RANTES, TSLP and CD40 ligand for the detection of inflammation-related platelet activation, e.g. in a biological sample.

In particular, the biological sample is as defined above in the “Sample” section.

A particular subject matter of the present invention is the use as defined hereinabove wherein the quantity, concentration and/or the proportion of the biomarkers AKT, PKC, CD62P, CD63, RANTES, TSLP and CD40 ligand is measured.

Measuring the quantity, concentration and/or the proportion of the biomarkers AKT, PKC, CD62P, CD63, RANTES, TSLP and CD40 ligand is in particular performed as described hereinabove in the section “Method for detecting a platelet inflammation”.

A particular subject matter of the present invention is the use as defined hereinabove, wherein is determined whether at least three of the biomarkers AKT, PKC, CD62P, CD63, RANTES, TSLP and CD40 ligand are activated or if at least five of the biomarker AKT, PKC, CD62P, CD63, RANTES, TSLP and CD40 ligand are not activated.

In particular, determining whether a biomarker is activated is performed as described hereinabove in the section “Method for detecting a platelet inflammation”.

If e.g. at least three of the biomarkers AKT, PKC, CD62P, CD63, RANTES, TSLP and CD40 ligand are activated, an inflammation-related platelet activation is detected.

If e.g. at least five of the biomarkers AKT, PKC, CD62P, CD63, RANTES, TSLP and CD40 ligand are not activated, no inflammation-related platelet activation is detected.

Other features and advantages of the invention will become clearer from the following examples, which are given only as non-limiting illustration.

### A BRIEF DESCRIPTION OF THE SEQUENCE LISTING

The SEQ ID NO: 1 corresponds to the SFLLRN TRAP peptide.

The SEQ ID NO: 2 corresponding to a AYPGKF PAR-4 agonist.

### FIGURES

FIGS. 1 to 8 show the variables in the order of decreasing magnitude (as estimated by a criterion of mean decrease of precision) in every model, with the variable on the ordinate and the magnitude on the abscissa.

FIG. 1 shows the variables in order of decreasing magnitude in the overall class.

FIG. 2 shows the variables in order of decreasing magnitude in the overall class corresponding to the absence of stimulation.

FIG. 3 shows the variables in decreasing order of magnitude in the class corresponding to ADP stimulation.

FIG. 4 presents the variables in order of decreasing magnitude in the class corresponding to collagen stimulation.

FIG. 5 presents the variables in decreasing order of magnitude in the class corresponding to fibrinogen stimulation.

FIG. 6 shows the variables in decreasing order of magnitude in the class corresponding to PAR-1 agonist stimulation.

FIG. 7 shows the variables in decreasing order of magnitude in the class corresponding to PAR-4 agonist stimulation.

FIG. 8 presents the variables in order of decreasing magnitude in the class corresponding to soluble CD40 stimulation.

### EXAMPLES

Equipment and Method

(i) Platelet-Rich Plasma

Peripheral blood samples from healthy individuals (n=10) were collected and placed in endotoxin-free tubes containing 3.2% sodium citrate (Vacutainer®, Becton Dickinson, San Jose, Calif.). Informed consent of blood donors was obtained prior to blood collection in a regional blood establishment, in accordance with French legislation. The blood samples were centrifuged at 192 g for 10 minutes at room temperature so as to obtain a platelet-rich plasma.

(ii) Stimulation of Platelets

The platelet response tests were carried out using a PAR-1 agonist (TRAP for Thrombin Receptor Activating Peptide SEQ ID NO: 1 SFLLRN, 6 μM) (Sigma-Aldrich, Saint Quentin-Fallavier, France), a PAR-4 agonist (AYPGKF, 200 μM) (Sigma-Aldrich, Saint Quentin-Fallavier, France), ADP (10 μM), collagen (50 μg/ml), sCD40L (50 ng/ml) or fibrinogen (50 μg/ml).

To this end, platelets from 10 donors were subject to each of the seven conditions (non-stimulated, PAR-1 agonist, PAR-4 agonist, ADP, collagen or sCD40L) and the response of 47 biological parameters was measured, namely:
the activation of membrane biomarkers: CD62P-%, CD62P-MFI, CD63-%, CD63-MFI,
the activation of soluble biomarkers: BCA-1, 6Ckine, TSLP, IL-33, GRO alpha, IFNgamma, MDC, CD40 ligand, CXCL9/MIG, CCL19/MIP-3b, CCL20/MIP-3a, serotonin, RANTES, soluble CD62, and
the activation of intracellular biomarkers: IKBa, IKKa, NFKB1, PKC, AKT, SYK(pY629/30), Axl(pY859), Lyn Total, Lyn Y937, PI3K110, SYK Py323, SYK total, ERK1/2 pY204/187, Gab1pY285/307/317, Gab2 pY614, PLCg1 pY1253, PLCg2 pY1197/1217, SHC pY349/350, STAT3 pY705, CBL pY700/731/774, WASH pY291, TEC pY206, RAP GEF1 GRF2 pY504, JAK3 pY785, Gab2 pY584, Gab2pY266, PTEN total, and Axl(pY772).

(iii) Quantification of Membrane Biomarkers

Platelet suspensions were incubated with a suitable monoclonal antibody, recognizing membrane proteins expressed on the surface of platelets, for 30 minutes at room temperature in the dark and then washed once with a phosphate saline buffer (X1).

Since all platelets constitutively express CD41a, said marker (recognized by a specific antibody conjugated to a fluorochrome—such e.g. as fluorescein isothiocyanate) was used for defining the window corresponding to platelets in flow cytometry analyzes.

Activated platelets are characterized by the expression of CD62P and CD63, among other markers. Monoclonal (or polyclonal) antibodies conjugated to a fluorochrome—such as e.g. allophycocyanin or phycoerythrin—directed against human CD62P and CD63 were then used for defining the windows of the cell population to be characterized as a function of the activation thereof.

The flow cytometry analyzes were performed using a FACSVantage SE apparatus equipped with CellQuestS-Pro software (BD Biosciences, Le Pont de Claix, France).

The results were given in MFI (Mean Fluorescence Intensity) or in percentage (%). The CD62P and CD63 immunolabeling makes it possible to specify the percentage of activated cells among the cells comprised in the window of analysis.

(iv) Quantification of Soluble Biomarkers

For every condition, the protein content of the a granules of the platelet supernatants was quantified using the Luminex® technology comprising magnetic beads specific for various human cytokines and chemokines (reference test HCYTOMAG-60K, HCYP2MAG-62K and HTek MAG-63K, Millipore, Molsheim, France). A molecule of canonical δ granule, namely serotonin, was quantified by ELISA (Enzyme-Linked Immunosorbent Assay) (IBL International, Hamburg, Germany). The absorbance at 450 nm (or 405 nm for serotonin) was measured using an ELISA plate reader (Magellan Software, Sunrise™, Tecan Group Ltd., Lyon, France). The results were normalized to pg/109 platelets/ml.

(v) Quantifications of Intracellular Biomarkers

For every condition, intracellular platelet proteins were extracted using the MILLIPLEX MAP EpiQuant sample preparation kit at a rate of 3×10⁷ platelets/ml lysis buffer, as per the manufacturer's instructions. Phosphorylated proteins were quantified using the MILLIPLEX MAP EpiQuant technology with the following five panels: MPEQMAG-100K, 102K, 103K, 104K and 110K (MILLIPORE). The results were expressed in pM per 3×10⁷ platelets/ml.

Results

(i) Identification of a Panel of Biomarkers for Detecting an Inflammation-Related Platelet Activation

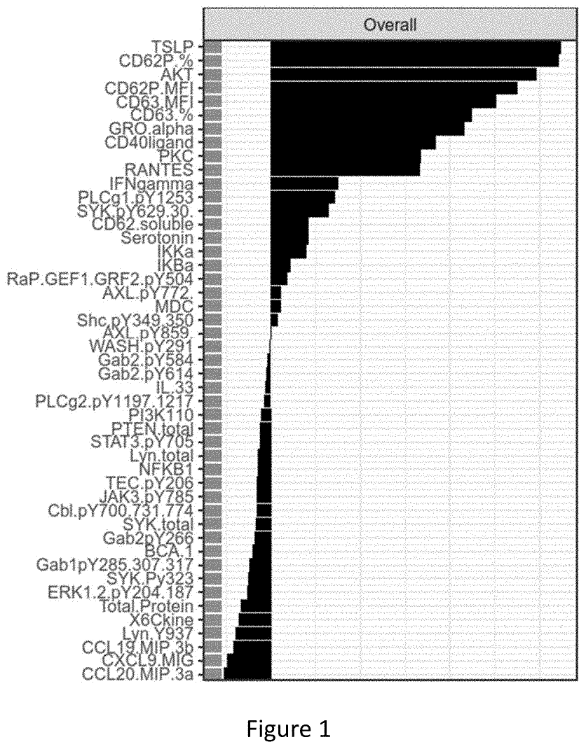

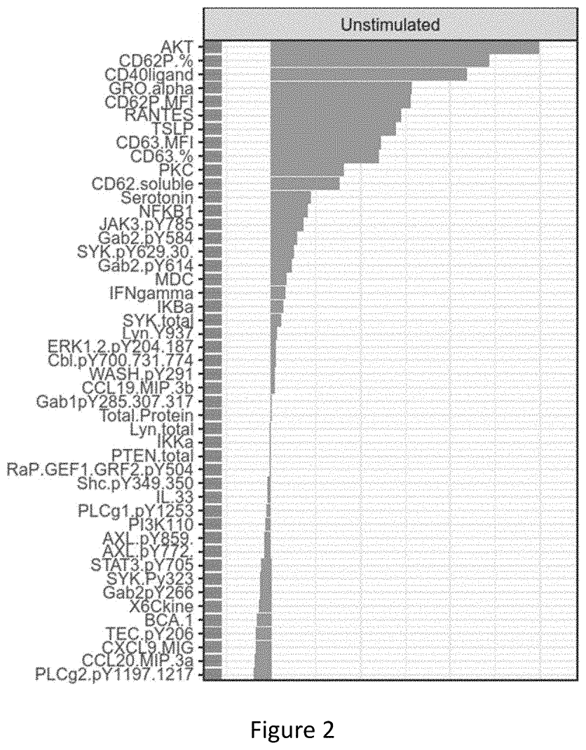

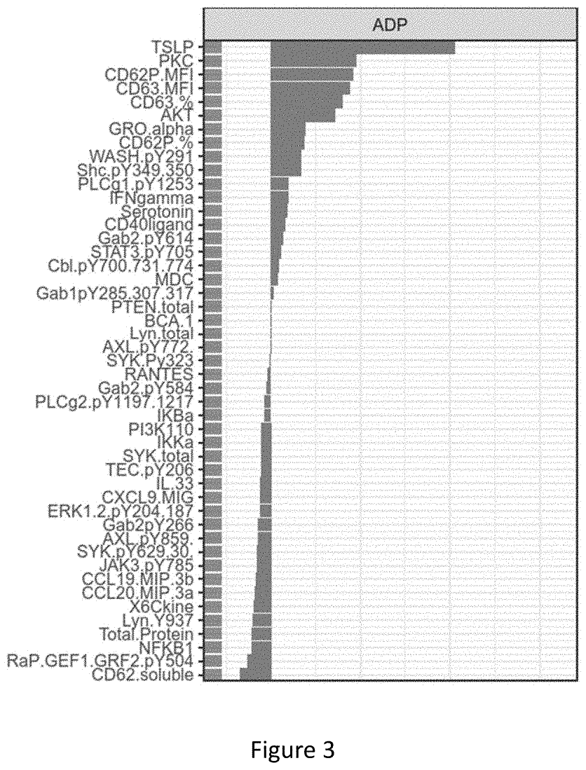

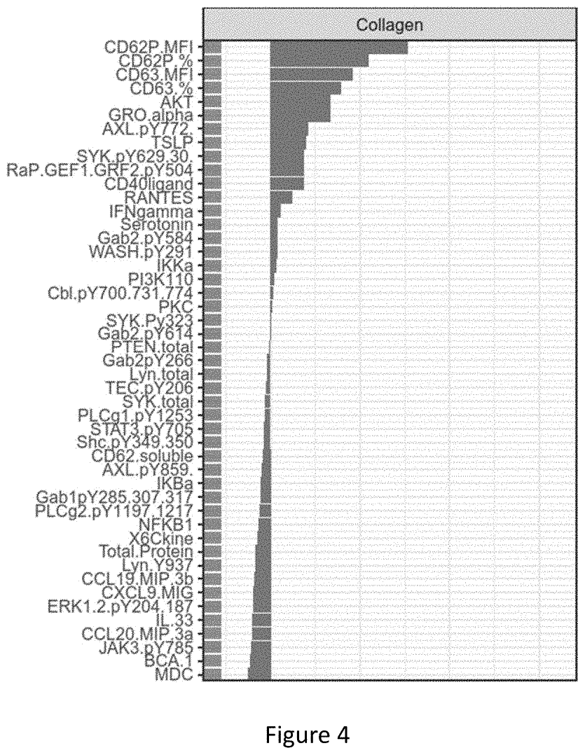

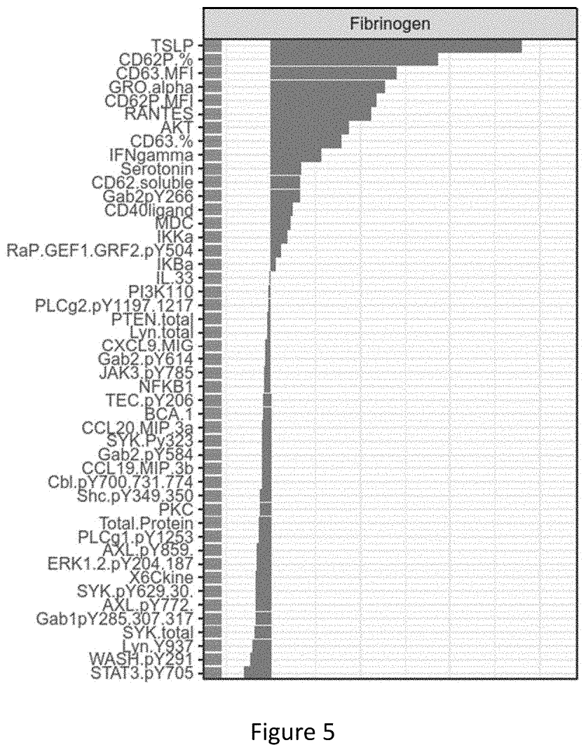

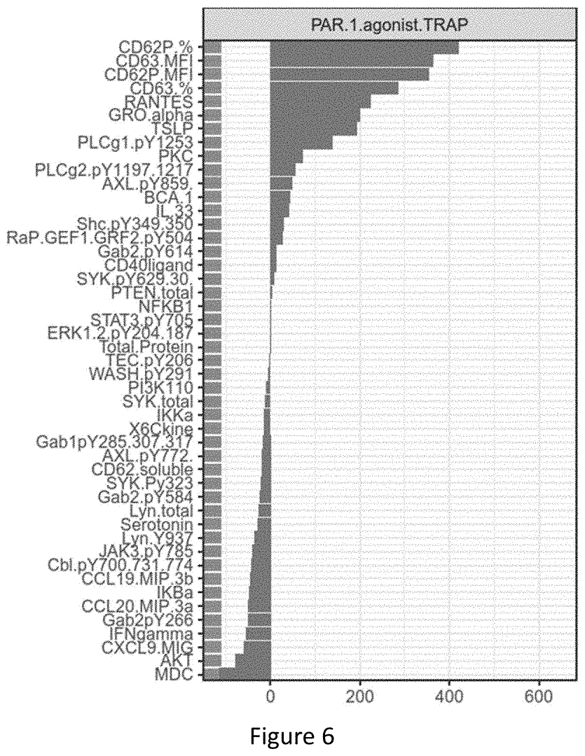

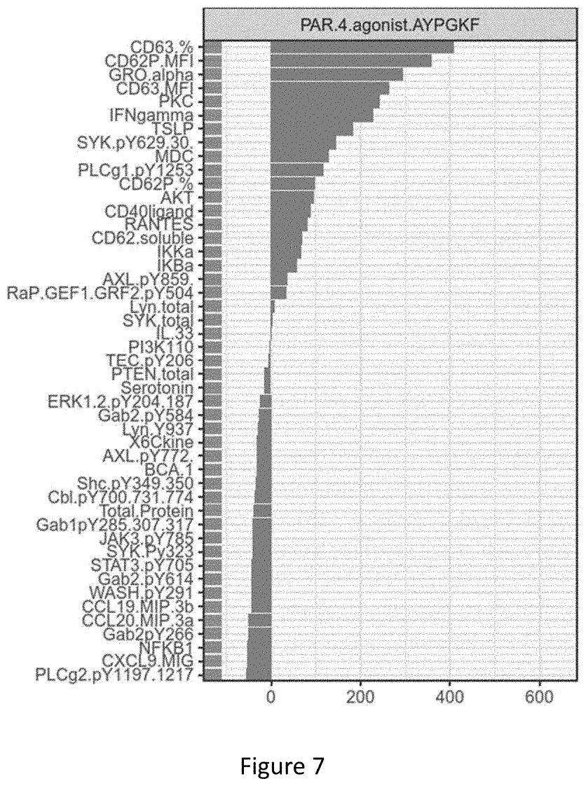

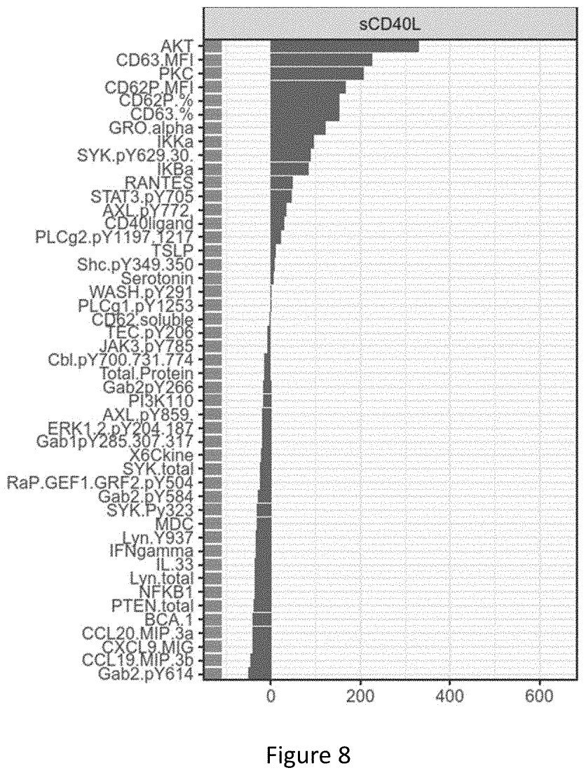

FIGS. 1 to 8 show the variables in order of decreasing magnitude (as estimated by a criterion of decrease of the mean precision) for the overall multi-class model (cf. FIG. 1) and for each individual class (cf. FIGS. 2 to 8).

Repeated cross-validations were performed for each of the 1023 sets of variables, based on the 10 most significant variables identified in FIG. 1. The results are shown in the Table 1 below.

TABLE 1
The best biomarker panels in terms of overall precision

| N | P | AKT | sCD40L | CD62P-MFI | CD62P-% | CD63-MFI | CD63-% | GROα | PKC | RANTES | TSLP |
|---|---|---|---|---|---|---|---|---|---|---|---|
| 8 | 87.9 | x | | | x | x | x | x | x | x | x |
| 8 | 87.6 | x | x | x | x | | x | | x | x | x |
| 7 | 86.8 | x | x | x | x | | | | x | x | x |
| 8 | 86.5 | x | | x | x | x | | x | x | x | x |
| 7 | 86.4 | x | | x | x | | x | | x | x | x |
| 7 | 86.4 | x | | | x | x | x | x | x | | x |
| 8 | 86.4 | x | x | x | x | | x | x | x | | x |
| 8 | 86.3 | x | | x | x | x | x | | x | x | x |
| 8 | 86.3 | x | x | x | x | | | x | x | x | x |
| 7 | 86.3 | x | x | x | x | | | x | x | | x |

N: Number of variables in the biomarker subset
P: precision in %

From the results presented in Table 1, a panel of biomarkers was defined comprising AKT, PKC, CD62P, CD63, RANTES, TSLP and CD40 ligand which allow the presence of an inflammation-related platelet activation to be determined with very high precision.

(ii) Identification of Specific Biomarkers for Activation of an Intra-Platelet Signaling Pathway

The biomathematical analysis has also identified specific biomarkers for the activation of an intra-platelet signaling pathway, such as:
NFKB1 and SHC pY349/350, associated with a stimulation by the PAR-1 agonist;
SYK (pY629/30), associated with a stimulation by the PAR-4 agonist;
BCA-1, CD63-%, Gab2 pY614, IFNγ and MDC, associated with a stimulation by the PAR-1 agonist and the PAR-4 agonist;
PKC, associated with a stimulation by the PAR-4 agonist and ADP;
CD63-MFI, associated with a stimulation by the PAR-1 agonist, the PAR-4 agonist and collagen;
soluble CD62 and CD62P-MFI, associated with a stimulation by the PAR-1 agonist, the PAR-4 agonist, collagen and ADP;
CD62P-%, GROα and RANTES, associated with a stimulation by the PAR-1 agonist, the PAR-4 agonist, collagen, ADP and sCD40L;
TSLP, associated with a stimulation by fibrinogen, the PAR-4 agonist, collagen, sCD40L,
AKT, associated with a stimulation by sCD40L, the PAR-1 agonist, the PAR-4 agonist, fibrinogen, collagen and ADP, and
CDL40, associated with a stimulation by the PAR-1 agonist, the PAR-4 agonist, fibrinogen, collagen and ADP.
(cf. table 2 below).

TABLE 2
Biomarkers for which a specific activation
was achieved by at least one activator

| | | | | fibrin- | col- | |
|---|---|---|---|---|---|---|
| | sCD40L | PAR-1 | PAR-4 | ogen | lagen | ADP |
| AKT | *** | *** | *** | *** | *** | *** |
| BCA-1 | | *** | ** | | | |
| CD40L | | *** | *** | *** | *** | *** |
| Soluble CD62 | | *** | *** | | *** | * |
| CD62P-% | ** | *** | *** | | *** | *** |
| CD62P-MFI | | *** | *** | | *** | *** |
| CD63-% | | *** | *** | | | |
| CD63-MFI | | *** | *** | | *** | |
| Gab2 pY614 | | * | * | | | |
| GROα | ** | *** | *** | | *** | *** |
| INFγ | | *** | *** | | | |
| MDC | | *** | *** | | | |
| NKFB1 | | * | | | | |
| PKC | | | *** | | | * |
| RANTES | * | *** | *** | | *** | *** |
| Shc pY349/350 | | * | | | | |
| SYK(pY629/30) | | | ** | | | |
| TSLP | ** | | *** | *** | *** | |

1. A method for detecting an inflammation-related platelet activation comprising measuring the quantity, concentration and/or the proportion of the biomarkers AKT, PKC, CD62P, CD63, RANTES, TSLP and CD40 ligand in a biological sample comprising platelets.

2. A method of diagnosis for inflammation-related platelet activation in an individual, comprising:
a) measuring the quantity, concentration and/or the proportion of the biomarkers AKT, PKC, CD62P, CD63, RANTES, TSLP and CD40 ligand in a biological sample comprising platelets of said individual,
b) comparing the results obtained during the step a) with a corresponding standard control value, and
c) deducing from the above if the individual has inflammation-related platelet activation.

3. The method according to claim 2, characterized in that the step c) comprises deducing therefrom whether a specific intra-platelet signaling pathway is activated.

4. A method for monitoring the efficacy of a curative or preventive treatment for inflammatory disease in an individual, comprising:
a) measuring the quantity, concentration and/or the proportion of the biomarkers AKT, PKC, CD62P, CD63, RANTES, TSLP and CD40 ligand in a biological sample comprising platelets of said individual at a time t during said treatment,
b) comparing the results obtained during the step a) with a corresponding standard control value and/or with a corresponding value obtained before the start of said treatment or at a time during the treatment, which is earlier than time t,
c) deducing therefrom whether the treatment is effective, and
d) optionally, repeating the steps a) to c).

5. A method of monitoring the evolution of inflammatory disease associated with inflammation-related platelet activation in an individual, comprising:
a) measuring the quantity, concentration and/or the proportion of the biomarkers AKT, PKC, CD62P, CD63, RANTES, TSLP and CD40 ligand in a biological sample comprising platelets of said individual at times t1 and t2, separated in time,
b) comparing the results at time t1 and time t2 obtained during the step a),
c) deducing therefrom whether the inflammatory disease evolves favorably, and
d) optionally, repeating the steps a) to c).

6. A stratification method for an individual with or at risk of suffering from inflammatory disease in a class of individuals with inflammation-related platelet activation or in a class of individuals without any inflammation-related platelet activation, comprising:
a) measuring the quantity, concentration and/or the proportion of the biomarkers AKT, PKC, CD62P, CD63, RANTES, TSLP and CD40 ligand in a biological sample comprising platelets of said individual,
b) comparing the results obtained during the step a) with a corresponding standard control value, and
c) deducing therefrom whether the individual belongs to the class of individuals with inflammation-related platelet activation or to the class of individuals without inflammation-related platelet activation.

7. The method according to claim 4, characterized in that said inflammatory disease is selected from the group consisting of atherosclerosis, pulmonary inflammation, rheumatoid arthritis (RA), inflammatory bowel disease (IBD), sepsis, severe sepsis, septic shock, cancer and combinations thereof.

8. The method according to claim 1, characterized in that the biological sample is a platelet-rich plasma sample.

9. A kit suitable for detecting an inflammation-related platelet activation comprising means for detecting at least seven biomarkers, characterized in that said at least seven biomarkers are AKT, PKC, CD62P, CD63, RANTES, TSLP and CD40 ligand.

10. A method for the detection of an inflammation-related platelet activation comprising the use of a kit according to claim 9.

11. A method for the detection of an inflammation-related platelet activation comprising the use of a biomarker panel including AKT, PKC, CD62P, CD63, RANTES, TSLP and CD40 ligand.

12. The method according to claim 5, characterized in that said inflammatory disease is selected from the group consisting of atherosclerosis, pulmonary inflammation, rheumatoid arthritis (RA), inflammatory bowel disease (IBD), sepsis, severe sepsis, septic shock, cancer and combinations thereof.

13. The method according to claim 6, characterized in that said inflammatory disease is selected from the group consisting of atherosclerosis, pulmonary inflammation, rheumatoid arthritis (RA), inflammatory bowel disease (IBD), sepsis, severe sepsis, septic shock, cancer and combinations thereof.

14. The method according to claim 2, characterized in that the biological sample is a platelet-rich plasma sample.

15. The method according to claim 3, characterized in that the biological sample is a platelet-rich plasma sample.

16. The method according to claim 4, characterized in that the biological sample is a platelet-rich plasma sample.

17. The method according to claim 5, characterized in that the biological sample is a platelet-rich plasma sample.

18. The method according to claim 6, characterized in that the biological sample is a platelet-rich plasma sample.

19. The method according to claim 7, characterized in that the biological sample is a platelet-rich plasma sample.


SEQUENCE LISTING (2 sequences)

SEQ ID NO: 1 - protein, 6 aa, Artificial Sequence
  TRAP
SFLLRN

SEQ ID NO: 2 - protein, 6 aa, Artificial Sequence
  agoniste de PAR-4
AYPGKF